(19) United States
(12) Patent Application Publication
(10) Pub. No.: US 2005/0252663 A1
(43) Pub. Date: Nov. 17, 2005

(54) Fiber-optic based automatic fire-suppression controller

(76) Inventors: Mark Petrus Olson, Plainwell, MI (US); Richard Lee Hilderbrandt, Martinsville, IL (US)

Correspondence Address: Mark P. Olson, 939 East Bridge Street, Plainwell, MI, 49080, US

(21) Appl. No.: 10/847,489

(22) Filed: May 17, 2004

(51) Int. Cl. A62C 35/00; A61L 9/04; A24F 25/00; G01J 1/04

(52) U.S. Cl. 169/5; 169/16; 169/17

(57) ABSTRACT

An Electro-mechanical device utilizing a fiber optic based sensor (Fiber Optic strand) for the purpose of detecting and extinguishing a fire momentarily after ignition. A light signal transmitted through the sensor (Fiber Optic strand) is used as a means to urge a spring loaded valve (Firing Valve) against its established position, thus blocking the dispersal of fire suppressing agents. This condition will persist until the sensor (Fiber Optic strand) is damaged by the presence of open flame in close proximity.

### CROSS-REFERENCE TO RELATED APPLICATIONS

Not applicable.

### BACKGROUND

1. Field of Invention

This invention relates to fire extinguishing systems, specifically those that do not require monitoring or operating personnel.

2. Description of Prior Art

Fire extinguishers and fire-suppression systems are widely used in both residential and industrial constructions. The effort to halt or minimize the huge annual losses to life and property has been ongoing for decades. Both extinguishers and systems are numerous and varied. They range from simple hand-held units to vast built-in networks.

Modern designs are for the most part both obvious and familiar. One innovation has been the incorporation of Fiber optic cable as a sensor. The light transmission properties of Fiber optic cable make an ideal sensor. There are several prior art references to this, being; U.S. Pat. No. 6,044,913 to Stehling, et al (2000), U.S. Pat. No. 6,164,383 to Thomas (2000), U.S. Pat. No. 4,227,577 to Iida (1980), U.S. Pat. No. 4,159,744 to Monte, et al (1979), U.S. Pat. No. 6,153,881 to Castleman (2000), and U.S. Pat. No. 6,111,511 to Sivathanu, et al (2000).

All of these examples make use of the light transmission properties of Fiber optic cable. Each of these systems is designed to control a conventional extinguisher. By measuring either light intensity or frequency, these designs seek to be more accurate in the detection of fire. These systems are fairly complex and have yet to become available to the general public.

A variant of these designs is described in U.S. Pat. No. 5,144,125 to Carter, et al (1992). Another property of Fiber optic cable is its susceptibility to heat damage. The light transmission property changes drastically as the cable is heated and then ultimately damaged. This design makes use of this change to allow for the tracking of a fire's progress, primarily in the lower levels of ships. This design is also complex and does not lend itself to use by the general public.

In each of the aforementioned designs, there is a singular, glaring drawback. The general public has put none of these designs into use. The majority of annual loss to fire, be it to life or property, occurs in home and business. The numerous disadvantages of these designs may well explain this:

(1) The aforementioned designs are, as a whole, fairly complex. This tends to make any one of them difficult and therefore expensive to manufacture. This in turn makes the finished product somewhat cost prohibitive to the general public. Furthermore, the more complex the system, the greater the odds of premature failure.

(2) Light sensors utilized in a contaminated and somewhat hostile environment, such as cooking facilities, machining operations, and heating facilities require lens covers of some type to prevent damage. Contaminants will impair the sensor or cause false alarms without regular and dedicated maintenance.

(3) Light sensors must be aimed at a fire to detect it. A design requiring multiple sensors multiplies the aforementioned disadvantages one for one. Fire, by nature, can begin anywhere that heat, oxygen, and fuel exist together. Therefore, a single light sensor is severely limited and ineffective.

(4) A design that tracks the progress of fire through enclosed areas is of little use if it cannot act upon the fire where it exists. The key to fire fighting is to attack the fire as soon as it is detected. In order to incorporate this maxim with the tracking design, an elaborate and expensive computer system would need to be linked.

### SUMMARY

The presented invention eliminates or lessens the impact of the aforementioned negatives through simplicity of design and use. The proposed device consists of a single closed loop of unshielded fiber optic strand, a photo cell assembly, and a pair of relay operated valves.

### OBJECTS AND ADVANTAGES

There are several objects and advantages to the present invention. They are:

(1) To provide a far simpler controller than those previously noted. This Fiber Optic Based Fire Suppression Controller (hereinafter referred to as “Controller”) lends itself to ease of manufacture and therefore, a finished good cost that allows the general public access to it.

(2) To provide a controller that is more reliable and is less subject to premature failure.

(3) To provide a controller that is not subject to contamination by airborne particles, condensation, or mishandling.

(4) To provide a controller that can directly attack a fire the instant it is detected.

(5) To provide a controller that requires little or no maintenance.

(6) To provide a controller that may be installed in a host of applications and can be so installed with relative ease.

Further objects and advantages are to provide a controller that requires relatively little space, can monitor a wide range of spaces, is non-obtrusive, can be modified to utilize any extinguishing medium, and can be adapted to numerous applications. A consideration of the ensuing description and drawings will make apparent further objects and advantages.

### DRAWING FIGURES

In the drawings, both a standard electronic schematic and simplified block diagrams are included.

FIG. 1 is the first section of the schematic of the operational prototype.

FIG. 2 is the second section of the schematic of the operational prototype.

FIG. 3 is a block diagram of the mechanical arrangement of the Loading Valve and the Firing Valve.

FIG. 4 is an electrical block diagram of the Fiber Optic Sensor circuit.

FIG. 5 is an electrical block diagram of the A/C and D/C Power Supplies.

FIG. 6 is a physical layout of the working prototype.

### REFERENCE NUMERALS IN DRAWINGS

R1, Resistor, 50 Ohm, 50 Watt
R2, Resistor, 50 Ohm, 50 Watt
R3, Resistor, 100 Ohm, 10 Watt
R4, Resistor, 50 Ohm, 10 Watt
R5, Resistor, 50 Ohm, 10 Watt
R6, Resistor, 10,000 Ohm, 0.25 Watt
R7, Resistor, 1,000 Ohm, 0.25 Watt
R8, Resistor, 82 Ohm, 0.25 Watt
C1, Capacitor, 0.1 Mfd.
C2, Capacitor, 0.1 Mfd.
Q1, Transistor, Phototransistor
Q2, Transistor, 2N2222
D1, Diode, 1N4004
LED 1, LED, Superbright, Red
LED 2, LED, Red
Z1, Rectifier, Full Wave, Solid State, 50 VAC
Z2, Rectifier, Full Wave, Solid State, 50 VAC
PB1, Push button, 12 VDC
S1, Single Throw, 120 VAC
S2, Single Throw, Roller Switch
S3, Single Throw, Roller Switch
S4, Single Throw, Roller Switch
TB1, Tie-board, 240 VAC
T1, Transformer, Center tapped, 1:2, 120 VAC
F1, Fuse, 250 VAC, 3 Amp
F2, Fuse, 250 VAC, 3 Amp
K1, Relay, 12 VDC, 10 Amp
K2, Relay, Siemens, 12 VDC, 10 Amp
K3, Relay, Siemens, 12 VDC, 10 Amp
K4, Solenoid, Pontiac Coil, L-04PL012D-C, 12 VDC
K5, Solenoid, Pontiac Coil, L-04PL012D-C, 12 VDC
P1, Plug, 120 VAC, 24 VDC
Fiber Optic Strand, Poly-Optical Products, FBR, PCM-1120, 25′
Loading Valve, Manufactured, Prototype
Firing Valve, Manufactured, Prototype
Lever 1, Manual Arming Lever, Manufactured, Prototype

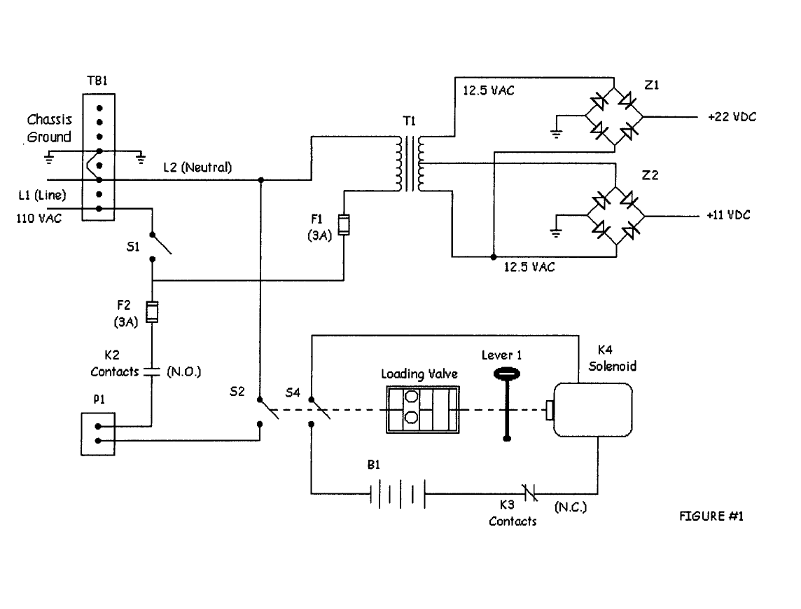

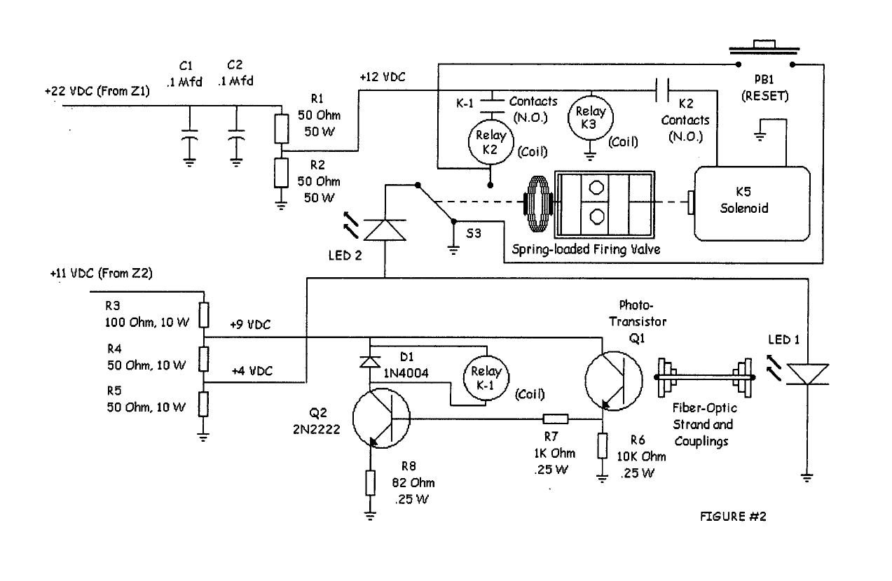

### DESCRIPTION—FIGS. 1 and 2, Preferred Embodiment

As the present invention is an Electro-mechanical system, a preferred embodiment is shown in FIGS. 1 and 2, that being a complete electrical schematic.

Commercially available 110 Volt A/C power is supplied to the system at TB1. 14 gauge, 2-conductor electrical cable with ground is required for a permanent installation of the present invention. The system power can then be manually controlled by externally mounted toggle switch, S1. Internal circuitry provides the necessary power needed to operate externally mounted Photo-transmitter LED 1 and externally mounted Phototransistor Q1. One end of a length of external unshielded Fiber Optic strand is centrally mounted directly in front of LED 1. The opposite end of the external Fiber Optic strand is centrally mounted directly in front of Q1. The sealed construction of these connections is such that contamination of the Fiber Optic strand is prevented. The Fiber Optic strand is then routed so as to be in close proximity of all anticipated fire sources.

Externally supplied Fire Extinguishing Medium is fed to the system at the inlet of the Loading Valve. The open position of the Loading Valve is manually selected by externally mounted Lever 1 and can only be selected once the system is “Armed and ready to fire”. The closed position of the Loading Valve can be manually operated or controlled by the system. The internal circuitry is so configured so that (1), in the event of commercial power failure or (2), if the system is switched off, internal battery B1 provides power to force the Loading Valve closed. This configuration provides both a means of safely and cleanly changing extinguishing mediums and prevents erroneous firing of the system due to commercial power loss.

The Firing Valve provides the final control of the Fire Extinguishing medium. By spring loading the Firing valve into the open position, then configuring the internal circuitry to pull and then hold the Firing Valve into the closed position, an “armed and ready to fire” condition is created. Only once this condition exists can the Loading Valve be moved to the open position, allowing the Fire Extinguishing medium to reach the inlet port of the Firing Valve. The system remains in this “Armed and ready to fire” condition provided that: (1) no fire is present in the proximity of the Fiber Optic strand, (2) commercial power is uninterrupted, or (3) the system is not manually turned off.

If the above mentioned condition (1) changes, that being a fire occurs in the proximity of (and thereby destroying) the Fiber Optic strand, the Firing Valve opens and allows the Fire Extinguishing Medium to reach externally mounted nozzles. The fire is thus directly attacked within seconds of ignition/detection.

If above mentioned condition (2) or (3) change, the Firing Valve will also operate as above. However, the Loading Valve will operate as previously mentioned, closing instantly and preventing an erroneous firing.

P1 is shown as an A/C power source to lights. Any single phase A/C power device can be powered via P1. If any of the above-mentioned conditions change, power is removed, thus preventing possible electrocution.

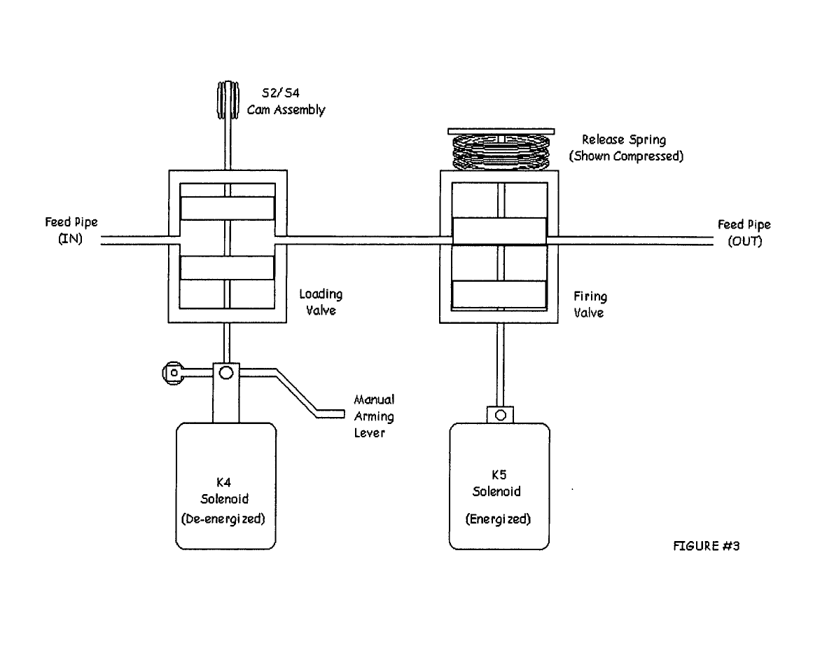

FIGS. 3-6, Additional Embodiments

Additional embodiments are shown in FIGS. 3, 4, 5, and 6. All of these figures are Block diagrams representing various mechanical and/or electrical configurations of the present invention.

FIG. 3 indicates the mechanical arrangement of both the Loading Valve and the Firing Valve including the electrical and mechanical positioning components. The figure indicates the “Armed and ready to fire” condition.

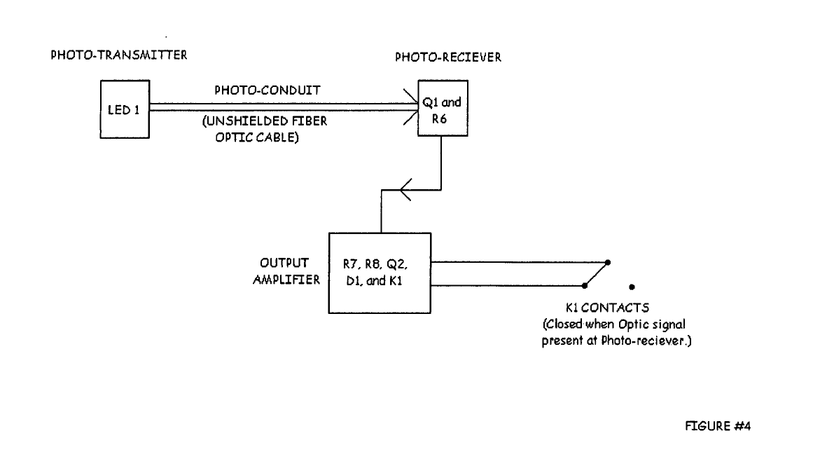

FIG. 4 indicates signal flow in the Fiber Optic sensor portion of the system.

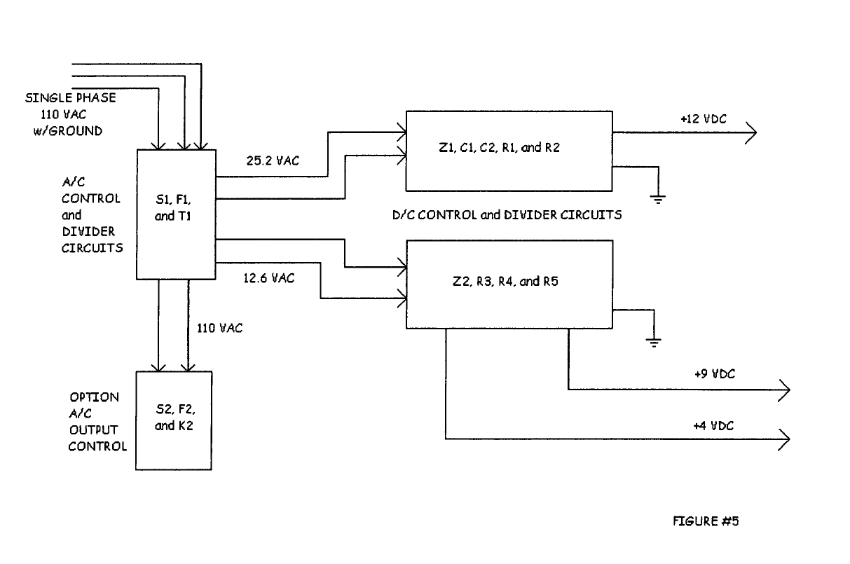

FIG. 5 indicates the system internal power supplies. Input and output voltages are noted as well as the related individual components.

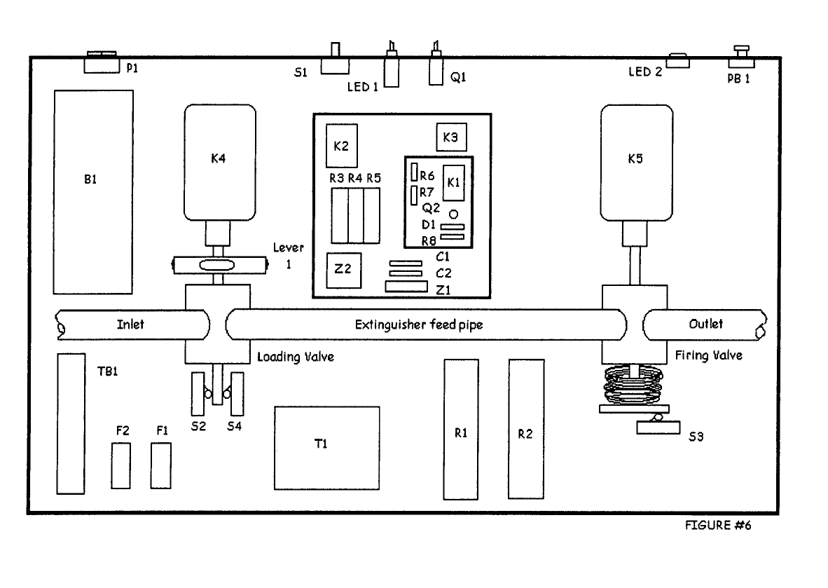

FIG. 6 indicates the component layout in the operational prototype.

Advantages

Based upon the above description, several advantages of our Controller are evident:

(a) The relative simplicity of the design allows for ease of manufacturability, therefore reducing finished good cost. Thus our Controller can be made readily available to the general public.

(b) Our Controller provides for ease of installation, operation, and maintenance.

(c) Our Controller may be installed virtually anywhere and provide total monitoring coverage while being relatively unobtrusive.

(d) Our Controller provides for unforeseen events such as commercial power failure or mishandling.

(e) Our Controller is unaffected by hostile environments or outside interference.

(f) Our Controller can utilize virtually any extinguishing medium.

(g) Our Controller provides for immediate fire suppression without monitoring equipment or personnel.

(h) Our Controller has the capability to remove electrical power from nearby equipment, thus averting possible electrocution hazards.

Operation

FIGS. 1, 2, 4, and 5.

NOTE: All voltages listed are measured under load.

Single phase A/C power is applied to the Controller at TB1 at pins 1 and 3 with ground provided at pin 5. A/C neutral is applied directly to the primary side of T1 and the normally open contact of S2. A/C line voltage is routed to S1. Closing S1 applies A/C line voltage to F1 and F2.

A/C line voltage through F1 is applied to primary side of T1. Stepped down A/C voltage from the center-tapped secondary of T1 is applied to solid state rectifiers Z1 and Z2. Full secondary voltage (25.2 VAC) is applied to Z1 while one-half secondary voltage (12.6 VAC) is applied to Z2. Rectifier Z1 converts secondary A/C voltage to +22 VDC. Filter capacitors C1 and C2 provide nominal filtering. A voltage divider consisting of R1 and R2 provide a standard relay drive voltage of +12 VDC. Rectifier Z2 converts secondary A/C voltage to +11 VDC. A voltage divider consisting of R3, R4 and R5 provide sensor circuit voltages of +9 VDC and +4 VDC.

A/C line voltage through F2 is applied to the normally open contacts of K2.

Relay drive voltage is applied to the normally open contacts of R1, a second set of normally open contacts in relay K2, and the coil of K3. With DC power return (Ground) hardwired to the coil of K3, the relay energizes, opening the normally closed contact in the Loading Valve circuit. This removes battery B1 from the Loading Valve circuit. The Loading Valve can now be manually operated.

The +4 VDC sensor circuit voltage is applied to the anodes of both LED1 and LED2. The cathode of LED1 is hardwired to ground and so illuminates. The intense red light from LED1 is transmitted through the Fiber Optic strand and returns to illuminate Q1. Ground to the cathode of LED2 is provided through the normally closed contact of S3. LED2 is the visual indicator that the Controller is disarmed (or has been fired).

The +9 VDC sensor circuit voltage is applied to the collector of Q1, across the cathode of D1, and to the coil of K1. Emitter bias for Q1 is provided from ground through R6. Providing that the Fiber Optic strand is intact, red light is transmitted to the base of Q1, causing the phototransistor to conduct.

Transistor Q2, resistor R7 and resistor R8 comprise a current driver switched on by the conduction of Q1. The coil of K1 is supplied a return to ground through the current driver circuit. Thus, as long as the Fiber Optic strand is intact, K1 will be energized. The biasing voltage for Q2 is set to a minimal value so that a small reduction in the amplitude of light received by Q1 will cause the current driver circuit to switch off. Diode D1 is configured across the coil of R1 so as to prevent “chattering” or the failure to de-energize when power is removed.

When control relay R1 is energized, +12 VDC is now applied through the closed contacts of R1 to the coil of holding relay K2. Depressing PB1 arms the Controller. This reset button momentarily supplies ground to the coil of K2. Provided that the sensor circuits are receiving the light signal, K2 will energize. Solenoid K5 now is supplied with +12 VDC through the closed contacts of K2. Solenoid K5 energizes and pulls the Firing Valve into the “armed and ready to fire” position (Closed). Switch S3 is mechanically operated by the Firing Valve and will change states when the “armed and ready to fire” position is reached. S3 now provides ground to the coil of K2, which remains after PB1 is released. Solenoid K5 is now holding the Firing Valve against a compressed spring and will continue to do so provided no interruption of the holding path created by R1, K2, S3, and the +12 VDC power supply occurs.

With relay K2 now energized, A/C line voltage is applied to P1. As LED2 is now off, the operator may proceed to manually operate the Loading Valve into the open position with the attached Lever 1. This allows the Fire Extinguishing Medium to travel through the piping to the inlet side of the Firing Valve. There it is held under pressure unless the Controller is fired. Switches S2 and S4 are mechanically operated by a cam physically attached to the Loading Valve. When the contacts of S2 are closed, A/C neutral is supplied to P1. A complete single-phase A/C power supply is now provided to operate external equipment that will be shut down in the event of the Controller firing.

When the contacts of S4 are closed, part of the battery supply circuit to solenoid K4 is completed. The circuit remains open so long as relay K3 remains energized. In the event of commercial power failure, relay K3 will de-energize, closing the normally closed contacts in the E1 battery circuit. Solenoid K4 now energizes, pulling the Loading Valve into the closed position, and thus shutting off the supply of Fire Extinguishing Medium. The cam is so constructed so that switches K2 and K4 remain closed until the Loading Valve is completely closed, at which time both sets of contacts will open. This both opens the battery power circuit to prevent battery drain and removes the A/C neutral leg from P1. As any interruption of the Firing Valve holding path will cause K2 to de-energize, commercial power failure will cause the contacts of K2 to open as well, removing the A/C line voltage from P1 as well.

### CONCLUSION, RAMIFICATION, AND SCOPE

It can be therefore determined that the relatively simple design of the Controller will allow for greater ease of manufacturability, thus reducing the finished good cost of the system, which will allow the general public access to purchase. Additionally, the flexibility of the Controller allows for usage in multiple applications, thus broadening marketability and the scope of intended use. Furthermore, the simple design of the Controller has the additional advantages in that

(1) rather than requiring highly trained service personnel, the layman can install and operate the Controller with relative confidence.

(2) repair can be easily effected in the event of damage or malfunction.

(3) it may be reused multiple times with minimal cost.

Although the description above contains many specificities, these should not be construed as limiting the scope of the invention but as merely providing illustrations of some of the presently preferred embodiments of this invention. For example, the Controller can be configured so as to operate on storage batteries for portability, miniaturized for single-use applications, be sealed and pressurized for unusual environments, etc.

Thus the scope of the invention should be determined by the appended claims and their legal equivalents, rather than by the examples given.

1. In a fire extinguishing system of the type comprised of an autonomous controlling device effecting the dispersal of flame suppressing agents, the improvement wherein said controlling device utilizes a closed loop of unshielded fiber optic strand as a sensor.

2. The sensor of claim 1 wherein said body of material is composed of plastic optical fiber.

3. The sensor of claim 1 wherein said body is a small diameter strand of variable length with unblemished termination at each end.

4. The sensor of claim 1 wherein said body is bereft of any armoring or shielding designed to prevent damage to said body.

5. The sensor of claim 1 wherein said body is ordered so that a light beam introduced into one said terminated end is transmitted through said strand and exits the remaining said terminated end uninterrupted.

6. The sensor of claim 1 wherein said body is so ordered that by heretofore described method said body directly effects dispersal of fire suppressing agent.
